(19) United States
(12) Patent Application Publication
(10) Pub. No.: US 2022/0037037 A1
(43) Pub. Date: Feb. 3, 2022

(54) TRACKING AND REDUCING INFECTION TRANSMISSION

(71) Applicant: Apollos Diagnostics, LLC, Bellevue, WA (US)

(72) Inventor: John Paul Alderete, III, Kapaa, HI (US)

(21) Appl. No.: 17/390,884

(22) Filed: Jul. 31, 2021

Related U.S. Application Data
(60) Provisional application No. 63/059,508, filed on Jul. 31, 2020.

(51) Int. Cl. G16H 50/80; G16H 50/30; G16H 70/60; G16H 50/20

(52) U.S. Cl. CPC G16H 50/80; G16H 50/20; G16H 70/60; G16H 50/30

(57) ABSTRACT

Apparatuses, components, devices, methods, and systems for tracking and reducing infection such as viral infection are disclosed. In particular, many of the examples in this disclosure relate to systems for determining when to test an individual, when to admit an individual to a group activity, and when to start or end a quarantine of an individual. Example systems may include a statistical model that determines risk levels for an individual and follow-up testing plans for individuals.

### CROSS REFERENCE TO RELATED APPLICATIONS

This application claims priority, as appropriate, to U.S. Ser. No. 63/059,508, titled “TRACKING AND REDUCING INFECTION TRANSMISSION” and filed Jul. 31, 2020, the disclosure of which is hereby incorporated by reference in its entirety.

### BACKGROUND

A virus is a submicroscopic infectious agent that replicates within a living host cell of another organism. When the host cell is infected by a virus, the virus causes the host cell to produce additional copies of the virus that may then infect other cells within the same organism or spread to other organisms.

When outside of a host cell, a virus exists as an independent particle that may be referred to as a virion. Typically, a virion will include genetic material enclosed in a capsid consisting of protein and, in some cases, a lipid bilayer. The genetic material may be a molecule of deoxyribonucleic acid (DNA) or ribonucleic acid (RNA).

Various types of viruses infect all types of life forms. Viruses are responsible for causing many diseases within the human species, including the common cold, influenza, chickenpox, rabies, acquired immune deficiency syndrome (AIDS), and many others. As another example, severe acute respiratory syndrome coronavirus 2 (SARS-CoV-2), the virus responsible for the 2019-20 coronavirus pandemic, has infected humans around the world, causing the coronavirus disease 2019 (COVID-19). The SARS-CoV-2 virus is also sometimes referred to as the 2019 novel coronavirus (2019-nCoV).

A virus may infect a host cell when a protein in its envelope binds to a receptor on the host cell. For example, the SARS-CoV-2 virus infects human cells when the spike (S) protein in the envelope binds to the angiotensin-converting enzyme 2 (ACE2) (ACE2 is also sometimes referred to as ACE-2 in scientific literature) receptor on human cells.

FIG. 1 is an illustration of the SARS-CoV-2 virus that is provided by the U.S. Center for Disease Control and created by Alissa Eckert, M S; Dan Higgins, MAMS. As can be seen in FIG. 1, the SARS-CoV-2 virus has a generally spherical envelope with numerous spike-like structures. These spikes are formed by the glycoprotein (a protein with carbohydrate groups attached to the polypeptide chain) known as the S protein of the SARS-CoV-2 virus. Other viral structural proteins include the envelope (E) protein and the membrane (M) protein in the envelope, and the nucleocapsid (N) protein, multiple copies of which may be bound to the RNA genome inside the envelope.

Once an organism is infected by a virus, it may release virion that can infect other organisms. For example, the organism may spread virion through coughing, sneezing, or even exhaling. Infected individuals may be asked or required to quarantine to prevent spreading of the virus to others. Unfortunately, quarantine is only effective in preventing the spread of virus by those individuals who have been identified as being infected.

Accurate and widespread testing is necessary to identify infected individuals so they may quarantine. Due to limits in testing resources, however, tests may only be available to a subset of individuals, such as those who are symptomatic, those who have recently traveled to hotspots (e.g., regions with a high number of known infections or a known presence of a specific variant), or those who are known to have had contact with infected individuals.

For some viruses, not all infected individuals will be symptomatic. Furthermore, some individuals may be capable of spreading the virus prior to onset of symptoms. Accordingly, if testing is provided based on severity of symptoms, infected individuals who are asymptomatic or have not yet developed symptoms that are severe enough to warrant testing may avoid quarantine and spread the virus because they were not tested. Furthermore, delays between test administration and test results may further allow infected individuals to spread the virus while waiting for test results.

Starting in 2020, many countries around the world instituted social distancing policies and quarantine policies designed to reduce the transmission of SARS-CoV-2. The social distancing policies aim to keep individuals far enough apart that virions shed by an infected individual are unlikely to come in contact with uninfected individuals. These social distancing policies come at great individual and societal cost as many common activities are prohibited.

The quarantine policies aim to reduce transmission of SARS-CoV-2 by isolating individuals who have tested positive for SARS-CoV-2, are suffering from symptoms that may be caused by SARS-CoV-2, or may have been exposed to SARS-CoV-2. In some jurisdictions, individuals arriving from outside of the jurisdiction may be required to self-quarantine for a specific duration of time (e.g., two weeks) after arrival in the jurisdiction. For example, the individual may be required to stay in a designated hotel during the quarantine time period. The quarantine may be enforced using a tracking device (e.g., a global positioning system (GPS) wrist/ankle band), smartphone tracking, or random in-person checks. Violations of the quarantine may, for example, result in the individual being denied access to the jurisdiction.

Like social distancing policies, these quarantine policies inflict high costs on individuals and society. The quarantine policies may specifically impact the tourism industry as many tourists may choose to avoid travel rather than endure a two-week quarantine. Effective testing, tracking, and statistical modeling may reduce the need for or duration of quarantine policies for at least some individuals, thereby limiting the potential negative impacts of these policies.

Although many of the examples in this disclosure relate to testing and tracking viral infections and more specifically, SARS-CoV-2 infection, aspects of this disclosure may be applicable to other types of infection, whether viral or caused by other types of pathogens which may infect humans or other organisms. For example, in addition to viruses, bacteria, parasites, and fungi may cause infection too.

### SUMMARY

In general terms, this disclosure is directed to testing and tracking of infections agents (or pathogens) and modeling transmission risk to determine quarantine and other policies for individuals. Many of the examples in this disclosure relate to systems for determining when to test an individual for infection, when to admit an individual to a group activity, and when to start or end a quarantine of an individual. Example systems may include a statistical model that determines risk levels for individuals and follow-up testing plans for individuals.

One aspect is an infection tracking system including: an evaluation session management engine that is configured to generate an evaluation session data structure and associate the evaluation session data structure with an individual; a test result processing engine that is configured to receive test results and associate the test results with the evaluation session data structure; and a risk assessment engine that is configured to determine a risk level for an individual associated with an evaluation session data structure.

Another aspect is a method of evaluating risk of infection transmission, the method comprising: receiving initial test results; associating the initial test results with a session data structure; evaluating a risk level based on the initial test results; and comparing the risk level to a threshold.

Yet another aspect is a non-transitory computer-readable storage medium comprising instructions stored thereon that, when executed by at least one processor, cause a computing system to perform a method comprising: generating a session data structure; associating the session data structure with an individual; receiving initial test results; associating the initial test results with the session data structure; determining a risk level based on the initial test results and the session data structure; comparing the risk level to a threshold; responsive to determining the threshold has not been satisfied, determining a follow-on testing plan; and transmitting a notification based on the follow-on test plan.

The details of one or more aspects are set forth in the accompanying drawings and description below. Other features and advantages will be apparent from a reading of the following detailed description and a review of the associated drawings. It is to be understood that the following detailed description is explanatory only and is not restrictive of the claims.

### BRIEF DESCRIPTION OF THE DRAWINGS

FIG. 1 is an illustration of SARS-CoV-2.

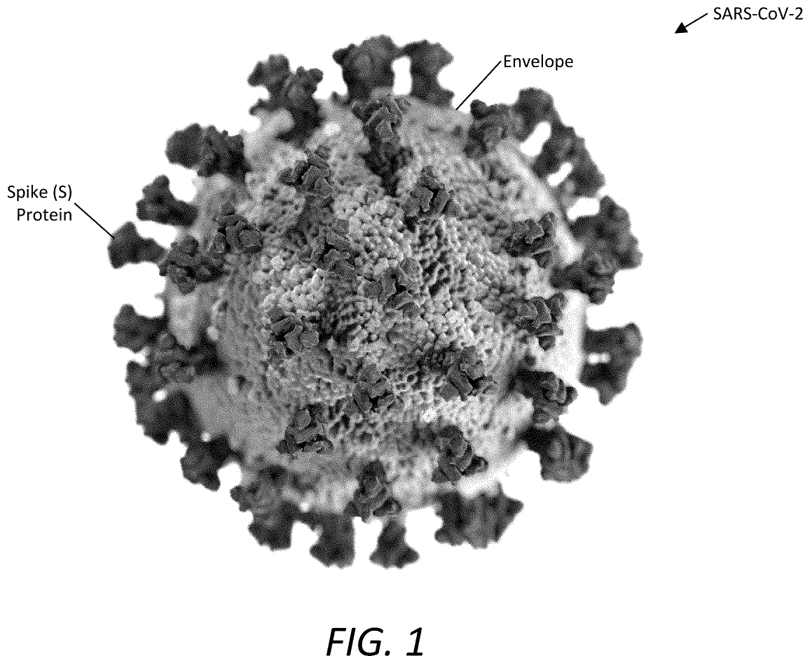

FIG. 2 is a schematic illustration of a system for determining event admission and quarantine requirements for individuals.

FIG. 3 is a schematic illustration of example data records usable to implement aspects of the present disclosure.

FIG. 4 is a diagram of an example method of evaluating risk of infection transmission and notifying of quarantine and admission determinations for individuals

FIG. 5 illustrates an example method of tracking infection so that an individual may participate in a group event using the system of FIG. 2.

FIG. 6 illustrates an example architecture of a computing device, which can be used to implement aspects according to the present disclosure.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The present disclosure relates to testing and tracking infection such as viral infection and to modeling risk of transmission of the infection. In particular, many of the examples in this disclosure relate to determining risk levels for transmission of SARS-CoV-2 based on results of testing individuals for infection by the SARS-CoV-2 virus. Individuals may be tested using lateral flow assay (also referred to as lateral flow immunochromatographic assay) technology or polymerase chain reaction (PCR) technology. The lateral flow assay technologies described herein may be used, as appropriate, with sandwich lateral flow assays or competitive lateral flow assays. Although many of the examples described herein relate to a lateral flow assay, it should be understood that the techniques may be used with other types of infection tests including but not limited to immunoassays such as enzyme-linked immunosorbent assays (ELISA), flow-through tests (immunoconcentration assays), aggregation assays, western blots, and other types of immunoassays.

The term “antibody,” as used herein, includes, but is not limited to a polypeptide substantially encoded by an immunoglobulin gene or immunoglobulin genes, or fragments thereof, which specifically bind and recognize an analyte (antigen or antibody). Examples include monoclonal, mixed monoclonal (which may function like defined polyclonal antibodies), polyclonal, chimeric, humanized, and single chain antibodies, and the like. Fragments of immunoglobulins include Fab fragments and fragments produced by an expression library, including phage display. See, e.g., Paul, FUNDAMENTAL IMMUNOLOGY, 3rd Ed., 1993, Raven Press, New York, for antibody structure and terminology.

The term “epitope” means an antigenic determinant capable of specific binding to an antibody. Epitopes usually consist of surface groupings of molecules such as amino acids or sugar side chains and usually have specific three-dimensional structural characteristics, as well as specific charge characteristics.

The terms “specifically binds to” and “specifically immunoreactive with” refer to a binding reaction that is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte. A variety of immunoassay formats may be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

A “protein” refers to a biopolymer composed of amino acid or amino acid analog subunits, which typically include some or all of the 20 common L-amino acids found in biological proteins. Although “protein” commonly refers to a relatively large polypeptide, e.g., containing 100 or more amino acids, and “peptide” to smaller polypeptides, the terms are used interchangeably herein. That is, the term protein may refer to a larger polypeptide, as well as to a smaller peptide, and vice versa.

An “immunologically reactive fragment” of a protein refers to a portion of the protein, which is immunologically reactive with a binding partner, e.g., antibody, which is immunologically reactive with the protein itself.

As used herein, a “receptor” of a protein refers to a portion of the protein which is reactive with a binding partner (e.g., an antibody, another protein, or a virus).

“Specificity”, as it relates to the testing technologies referenced herein, refers to the ability of the assay to specifically detect a specific infection such as SARS-CoV-2 infection.

“Reliability”, as it relates to the testing technologies referenced herein, refers to the percentage of a population that is detected as a true positive. For example, at least 80% reliability means that the assay will detect infection in at least 80% of the subjects tested who are in fact infected, as determined, for example, by a confirmatory test such as viral culture or PCR. The term is also used interchangeably with “sensitivity.”

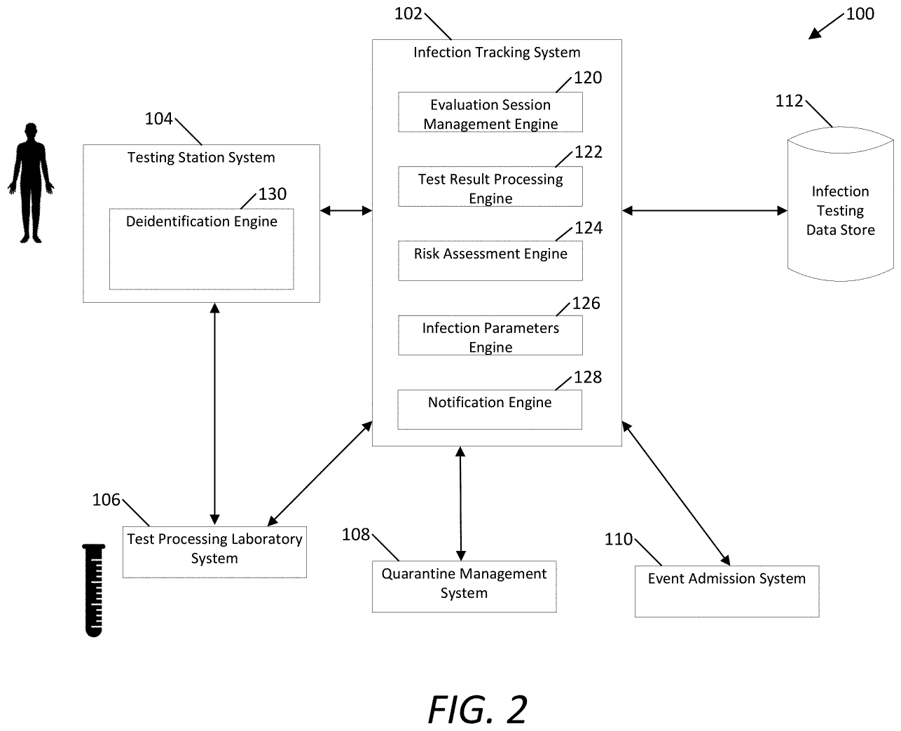

FIG. 2 is an example system 100 for event admission and quarantine requirements for individuals. The system 100 for may track and use testing data for one or more individuals to determine when an individual may be released from quarantine or whether to admit an individual to an event (e.g., an event in which risk of transmission is increased such as larger group gatherings and use of public transit).

In this example, the system 100 includes an infection tracking system 102, a testing station system 104, a test processing laboratory system 106, a quarantine management system 108, an event admission system 110, and an infection testing data store 112. Although this figure shows one testing station system and one test processing laboratory system, it should be understood that multiple testing station systems and multiple test processing laboratory systems may be part of (or interact with) the system 100.

The infection tracking system 102 is a system that tracks infection or infection risk for one or more individuals. The infection tracking system may, for example, be used within a geographic region (or jurisdiction) to reduce the risk of infection spreading from potentially infected individuals to non-infected individuals. In this example, the infection tracking system 102 includes an evaluation session management engine 120, a test result processing engine 122, a risk assessment engine 124, an infection parameters engine 126, and a notification engine 128.

The evaluation session management engine 120 manages evaluation sessions for an individual. For example, an evaluation session may be initiated based on data received from the testing station system 104 when an initial test is conducted on an individual. An initial test may be a first test conducted on an individual or a first test conducted on an individual after the individual has engaged in a specific activity such as travelling outside of a geographic region, interacting with another individual who is known to be infected, or attending a specific event with a large group of people.

In response to receiving the data, the evaluation session management engine 120 may generate a session data structure and associate that data structure with the individual. In some implementations, associating the data structure with the individual may include storing identifying information about the individual. Associating the data structure with the individual may also include storing a key or code that is associated with the individual, but not necessarily storing any identifying information about the individual.

The test result processing engine 122 receives test results from, for example, the test processing laboratory system 106. In response to receiving test results, the test result processing engine 122 may associate those test results with an evaluation session generated by the evaluation session management engine 120. The received test results may include an identifier that the test result processing engine 122 may use to associate the results with a session. As described further herein, the test result processing engine 122 may receive more than one test results that are to be associated with one evaluation session.

The test result may include a binary value indicating the presence or absence of the infection. The test result may also include one or more numeric values corresponding to a determined measurement related to the test. The test result may also include information about the type of test (e.g., antigen, PCR, or serologic), the manufacturer of the various test supplies and test equipment, batch or serial numbers for the various test supplies and test equipment, the location and date at which the test was administered, and identification information associated with the test administrator.

The risk assessment engine 124 determines a risk level for an individual associated with an evaluation session. The risk level may be determined based on various factors including, but not limited to, test result data and activities associated with the evaluation session and infection parameters. The risk level may also be determined based on factors associated with the individual, such as whether the individual has been vaccinated, date of vaccination, type of vaccine, etc. For example, the risk assessment engine 124 may determine a risk value for an individual who engaged in travel activity and then was tested twice following completion of the travel, once upon arrival and once six days after arrival. The risk assessment engine 124 may determine a numeric risk value that corresponds to the likelihood that an individual who has been infected receives one or more negative test results (e.g., negative test results twice when the tests were taken six days apart). As will be discussed further herein, the risk value may be based on various infection parameters such as the pre-contagious incubation period (i.e., the duration of time between being infection and testing positive, which may correspond to when the individual is contagious). Other infection parameters may also be used by the risk assessment engine 124.

The infection parameter engine 126 manages parameters associated with one or more infections. The infection parameter engine 126 may also allow a user of the system to modify parameter values or add new parameter values (e.g., by generating a user interface for modifying or adding values or providing an API to modify or add values). These parameter values may, for example, be used by the risk assessment engine 124. The infection parameter engine 126 may allow modifications of parameter values as new research provides a better understanding of a specific infectious agent.

Non-limiting examples of parameters managed by implementations of the infection parameter engine 126 include a pre-contagious incubation period, duration of infectivity, infection prevalence, and the effective reproduction rate. The pre-contagious incubation period refers to the duration of time between when an individual is infected and when that individual becomes infectious to others (contagious) (this is also when it is believed the individual will begin to test positive for infection). The duration of infectivity refers to the duration of time between when an individual is infected and when that individual ceases to be infectious to others (contagious). The effective reproduction rate refers to the average number of new infections generated by one infection.

The notification engine 128 generates and transmits notifications. For example, the notifications may be generated based on a risk value determined by the risk assessment engine 124. The notifications may, for example, be sent to individuals who have been tested and are currently in quarantine. The notifications may indicate that the quarantine can be terminated based on the risk value. The notification may also indicate that the individual should be tested on a specific date in order to potentially shorten the duration of quarantine. The notification engine 128 may, for example, transmit notifications via a computing network (e.g., email) or telephone network (e.g., SMS text messages).

The testing station system 104 transmits information about tests that are performed to the infection tracking system 102. The testing station system 104 may, for example, transmit information to the infection tracking system 102 that includes a date associated with the test, the type of test administered, who administered the test, the location at which the test was administered, and information to associate the test with a specific individual. In some implementations, the testing station system 104 may also transmit information about specific events or activities that the individual participated in. For example, the testing station system 104 may be located in an airport and may transmit information to the infection tracking system 102 about a flight an individual took.

The testing station system 104 may include a deidentification engine 130 that removes information that could directly identify the individual associated with a test. For example, the deidentification engine 130 may associate test results with an individual code assigned to an individual rather than with the individual's name. The individual may be given or required to wear a tamper-proof band that includes the code during a quarantine period. The testing station system 104 may, for example, transmit a test identification code and an individual code to the infection tracking system 102.

The test processing laboratory system 106 is a system that transmits test results to the infection tracking system 102. For example, the test processing laboratory system 106 may transmit the results along with a test identification code to the infection tracking system 102. The infection tracking system 102 may then use the test identification code to associate the test results with a session associated with an individual based on having previously received the same test identification code from the testing station system 104. In some implementations, the test processing laboratory system 106 may also transmit information about the supplies and equipment used to process the test such as serial numbers or batch numbers. This information may be used by the infection tracking system 102 to determine test specificity and sensitivity values. In some implementations, the information may also be used to disregard test results if a problem is later discovered with a specific piece of equipment or batch of supplies.

Although the testing station system 104 and the test processing laboratory system 106 are shown as separate components, these components may be combined. For example, when direct antigen testing is used, test results may be available immediately and a separate test processing laboratory may not be needed. In this case, the testing station system 104 may also operate as the test processing laboratory system 106 by, for example, transmitting test results to the infection tracking system 102. In some implementations, the testing station system 104 may include a portable device that processes a test sample from an individual to determine the presence of infection. The portable device may receive a sample directly and process the sample. The portable device may include a port for receiving a strip that includes the sample, such as a lateral flow assay test strip. The portable device may then read the strip (e.g., optically, magnetically, or otherwise) to determine the presence of infection. The portable device may then transmit the test results to the infection tracking system 102. The portable device may include a network interface component to directly access the internet to transmit results to the infection tracking system 102. In some implementations, the portable device may access the internet and communicate with the infection tracking system 102 using a nearby cellular device. For example, the portable device may include a Bluetooth component through which the portable device connects to and communicates with a nearby cellular device to transmit test results.

The quarantine management system 108 determines when an individual may terminate a quarantine. In some implementations, the quarantine management system 108 specifies a default quarantine duration (e.g., 14 days) that may be reduced based on the individual completing one or more tests and a risk value determined by the risk assessment engine 124 based on those tests. In some implementations, the quarantine management system 108 may compare the determined risk value to a specific threshold. If the risk value is below the threshold, the quarantine management system 108 may determine that the individual may terminate the quarantine.

The event admission system 110 determines when an individual may participate in an event. In some implementations, the event admission system 110 may compare a risk value determined by the risk assessment engine 124 to a specific threshold. If the risk value is below the threshold, the event admission system 110 may determine that the individual may be permitted to enter a venue or participate in an event. The threshold value may vary based on the type of event the individual is seeking to attend such as based on the number of people likely to be in attendance at the event and how much social distancing can be used at the event.

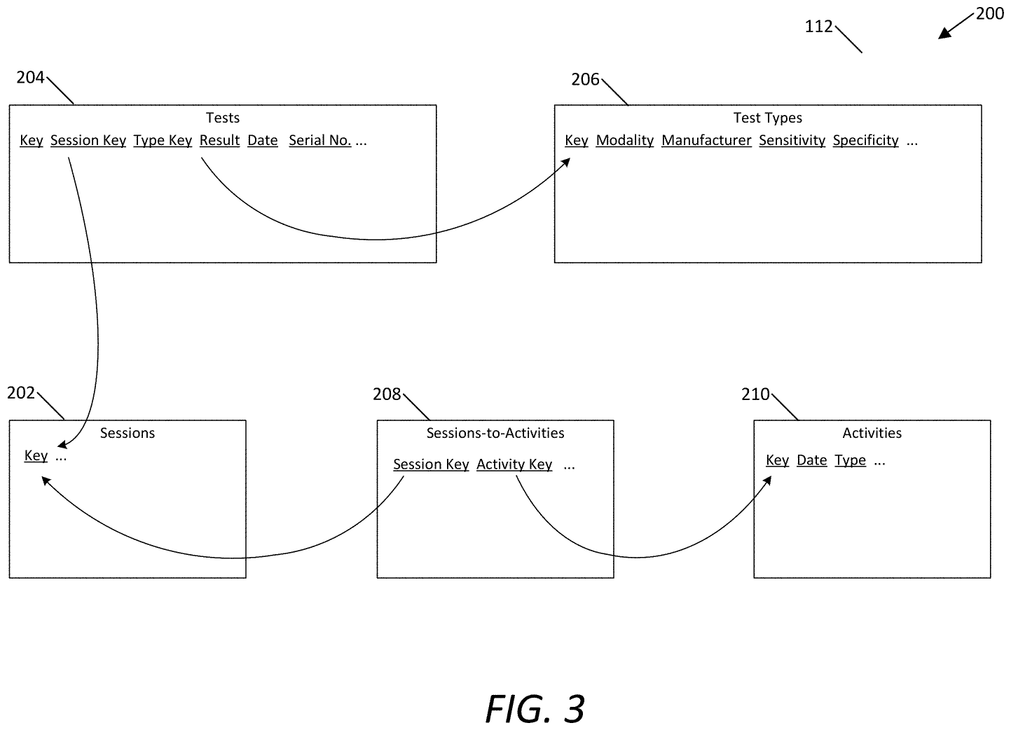

The infection testing data store 112 is an example data store that may store records related to individuals, activities, and testing that may be used by the system 100. Various components of the system 100 may read records from the infection testing data store 112 or update or write records to the infection testing data store 112. The infection testing data store 112 may include a relational database or a non-relational database. An example of data tables used to store records by embodiments of the infection testing data store 112 is illustrated and described with respect to FIG. 3.

FIG. 3 is a schematic illustration of an example data store 200 that includes example data tables usable to implement aspects of the present disclosure. These data tables may, for example, define records that are stored in the data store 200.

In this example, the data store 200 includes a sessions data table 202, a tests data table 204, a test types data table 206, a sessions-to-activities data table 208, and an activities data table 210.

In this example, the sessions data table 202 includes a key attribute for each record. The key attribute may be associated with an evaluation session for an individual. In some implementations, the sessions data table 202 may also include attributes that identify the individual associated with the evaluation sessions, such as one or more of a name, identifying number, or contact information. In some embodiments, the sessions data table 202 does not include any information that could be used to identify the individual associated with the evaluation session. Instead, for example, the individual may be given a record that includes the key associated with the session. A new record may be added to the sessions data table 202 by the evaluation session management engine 120 in response to receiving evaluation session initialization data from the testing station system 104.

The key attribute may be a primary key to the sessions data table 202. A primary key, for example, may be required (e.g., by a database management system of the data store) to have a unique value for each record in the sessions data table 202. In some implementations, a database management system may generate the values for the key attribute. The key attribute may be used in other data tables to refer to a session record (e.g., as a foreign key to the sessions data table 202).

In this example, the tests data table 204 includes a key attribute, a session key attribute, a type key attribute, a result attribute, a data attribute, and a serial number attribute. Some embodiments include more, fewer, or different attributes.

The key attribute may be a primary key to the records in the tests data table 204. The session key may be a foreign key to the sessions data table 202 that identifies a record in the sessions data table 202 to which the test relates (e.g., because the test was performed on the individual associated with the session). A foreign key attribute may store the value of a key attribute (primary key) of another table to identify a record in that table (e.g., the session key stores the value of a key attribute of a record in the sessions data table 202).

The type key may be a foreign key to the test types data table 206 to identify a data record with information about the type of test that was performed. The result attribute may store one or more Boolean, numeric, or textual values that represent the results of the test. The date attribute may store a value corresponding to the date the test was performed. Some implementations may also include an additional date attribute that stores a value corresponding to the date test results were determined (or received from the test processing laboratory system 106). The serial number attribute may store one or more values that identify specific test supplies or equipment used to perform a test. The serial number attribute may, for example, be used by the risk assessment engine 124 to adjust risks values based on information about specific test supplies and equipment such as recalls or contamination events.

In this example, the test types data table 206 includes a key attribute, a modality attribute, a manufacturer attribute, a sensitivity attribute, and a specificity attribute. Some embodiments include more, fewer, or different attributes.

The key attribute may be a primary key to the records in the test types data table 206. The modality attribute may store a value that identifies the type of test performed such as a PCR test, a direct antigen test, or a serology (antibody) test. The manufacturer attribute may store one or more identifiers of the manufacturer of the test equipment and supplies. In some implementations, additional values may be stored that identify the make/model type of the testing equipment and supplies. Some implementations may also store a validity attribute, that may indicate whether the test should be considered valid (e.g., by default the test may be considered valid but if a recall or other information comes to light, the test type could be marked invalid). A similar validity attribute may also be included as an attribute of the tests data table 204. The sensitivity value and the specificity value may store numeric values that correspond to the specificity and sensitivity determined for tests of the specific test type.

The sessions-to-activities data table 208 may be used to associate a session associated with an individual to activities that the individual has engaged in. In this example, the session-to-activities data table 208 includes a sessions key attribute and an activity key attribute. Some embodiments include more, fewer, or different attributes. The session key may be a foreign key that refers to a record in the sessions data table 202 and the activity key may be a foreign key that refers to a record in the activities data table 210. In this manner, the presence of a record having a specific session key and a specific activity key may indicate an association between the specified session and the specific activity (e.g., that the individual associated with the session participated in the activity).

In this example, the activities data table 210 includes a key attribute, a date attribute, and a type attribute. Some embodiments include more, fewer, or different attributes. The key attribute may be a primary key to the records in the activities data table 210. The date attribute may store a date that corresponds to the date the activity occurred. The type attribute may be a value that corresponds to the type of activity. The type value may be used by the risk assessment engine 124 to determine how risky participation in the activity is from an infection exposure perspective (e.g., outdoor activities may be less risky than indoor activities, etc.). The activities data table 210 may also store other information about the activity such as the duration of the activity.

In determining the risk value for an individual, the risk assessment engine 124 may query the sessions-to-activities table 208 to identify sessions associated with other individuals who participated/attended the same activity (e.g., were on the same flight). The results stored in test records associated with the sessions of those other individuals may impact the determined risk value (e.g., if someone else on the same flight tests positive, the risk value may be increased accordingly). In some implementations, the sessions-to-activities data table 208 may also store specific information about an individual's participation at an activity such as a seat number or location if that information is available. This information may be used by the risk assessment engine 124 to evaluate the risk of transmission from an individual who has tested positive for an infection to another individual who also participate in the same activity (e.g., the determined risk of transmission may be lower or higher based on proximity of the individuals).

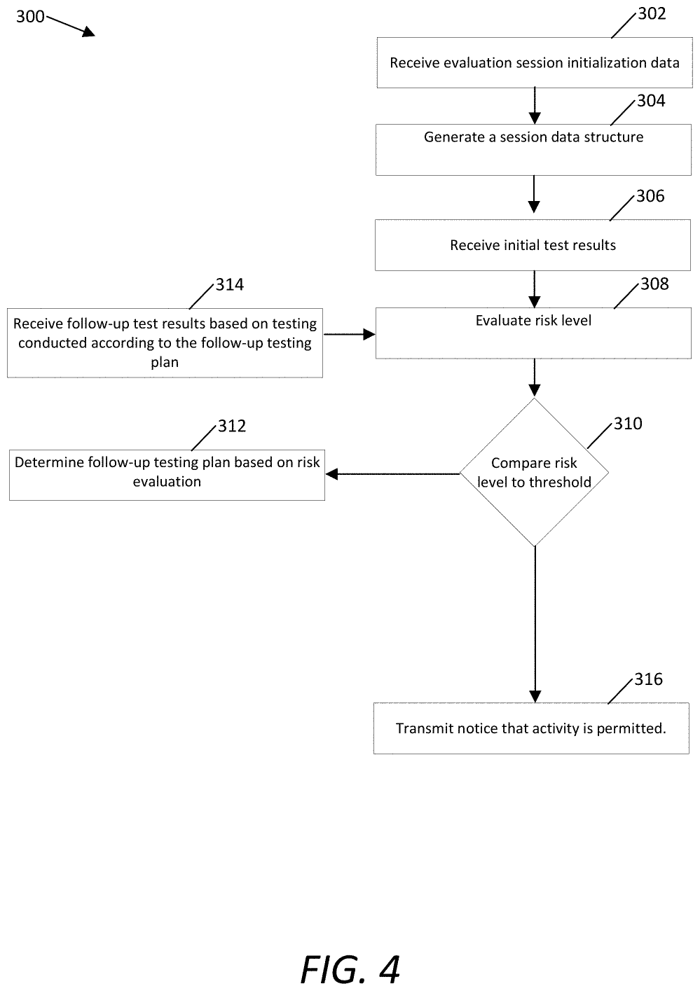

FIG. 4 is a diagram of an example method 300 of evaluating risk of infection transmission and notifying of quarantine and admission determinations for individuals, in accordance with implementations described herein. The operations described below may be performed by the infection tracking system 102 or various other components of the system 100.

At operation 302, evaluation session initialization data is received. The evaluation session initialization data may, for example, be received from the testing station system when an individual is initially evaluated. The initial evaluation may include conducting an initial infection test on the individual. The individual may be evaluated as part of a protocol to access a specific location or geography during an outbreak of an infection. For example, the initial evaluation may be part of a quarantine policy that applies to individuals entering a specific geographic location. The evaluation session initialization data may include an identifier that is usable to associate the evaluation session and test results with an individual. That identifier may include information about the individual, such as a name, government assigned identification number (e.g., social security number, passport number, driver's license number), phone number, or other contact information. In some implementations, the identifier may be associated with an individual without including any personally identifiable information. For example, the identifier may be a numeric value or code assigned to the evaluation session when the individual is initially evaluated. The individual may be given a wearable band that includes the code or may otherwise record the code. The band may be made of a tamper proof material so that it cannot be taken off and transferred to another individual. In some implementations, personally identifiable information may be stored for the individual in an encrypted format such as public key encryption. The code may serve as a private key that is usable to decrypt the encrypted information.

At operation 304, a session data structure is generated. For example, the session data structure may be similar to a row from the sessions data table 202 that is illustrated and described with respect to FIG. 3. In some implementations, the evaluation session management engine 120 may communicate with the infection testing data store 112 to generate the session data structure.

At operation 306, initial test results are received. The initial test results may, for example, be received from the test processing laboratory system 106. For example, if the initial test is a PCR test, the test will need to be processed using specialized test processing equipment that is typically available in a testing laboratory. The test results may also be received from the testing station system 104. For example, if the initial test is a direct antigen test, the test results may be available at the testing station. The test results may be transmitted to the infection tracking system 102 along with the code associated with the evaluation session (e.g., the individual may provide the code or a wearable band that includes the code).

At operation 308, a risk level is evaluated. The risk level may be determined by, for example, applying a statistical model to the testing and activity data associated with the session. For example, the risk level may correspond to the likelihood that the received test result (or results) would occur on tests of an infected individual who engaged in the activities recorded in the activities database. An example statistical model to evaluate risk level of an individual who has flown into a geographic location may use many factors. These factors may include many parameters that are managed by the infection parameters engine 126.

For example, the statistical model may use a base infection rate (e.g., corresponding to rate of infection in the geographic location from which the individual departed) and the duration of the pre-contagious incubation period of the specific infection. Some infectious agents may evolve over time to become more or less infectious and more or less lethal. The term variant is used herein to refer to different infectious agents that have evolved from another infection agent. The statistical model may also use the presence and prevalence of one or more variants. The variants may have different pre-contagious incubation periods or other properties.

As infections generally behave differently in different individuals, the duration of the pre-contagious incubation period used in the model may be an average value for a population. The model may also incorporate a standard deviation or variance value.

These factors may be used by the statistical model to, for example, determine the risk that the individual was infected shortly before departure and is currently harboring the infection but has not yet become contagious (and therefore possibly not yet testing positive). If this individual were admitted, there would be a risk that the infection would become contagious over time. If the individual has tested negative multiple times over a time period that lasts longer than the pre-contagious incubation period, that risk decreases.

The statistical model may also evaluate risks based on the activity or activities the individual engaged in prior to or after initiation of the evaluation session. For example, the session record may be associated with an activity record corresponding to the flight the individual traveled on to reach the geographic destination. In this case, the statistical model may also incorporate the risk that the individual was infected during the flight. That risk may be modeled based on many factors such as the duration of flight, the number of people on the flight, the capacity of the airplane, and the test results of others on the flight. If, for example, the infection testing data store includes test records for all individual who shared a flight and all those individuals tested negative after the flight, it may be determined that the risk of transmission is lower than if test results are not available for all individuals on the flight or if one or more individuals tests positive. The risk of transmission on a flight may also be adjusted based on proximity to an individual testing positive. Other factors that may be incorporated by the statistical model include the type of test performed, the sensitivity and specificity of the test performed, and the manufacturer of the test.

At operation 310, the risk level is compared to a threshold. The threshold level may be a parameter that is managed by the infection parameter engine 126. The threshold level may correspond to a determined acceptable level or risk that a governmental or private entity has selected to balance the risk of infection transmission against the societal and economic impact of, for example, completely prohibiting entry of those from outside a geographic area. In some implementations, the threshold may be selected such that the risk value calculated by the statistical model based on a single negative test result after a flight is insufficient to avoid quarantine (or similar protocols) but the risk value calculated by the statistical model based on multiple negative test results separated by a duration of time equal to or greater than the pre-contagious incubation period of the infection would meet the threshold. If the risk threshold is satisfied (i.e., the calculated risk value is below the threshold), the method continues to operation 316. Otherwise, the method continues to operation 312.

At operation 312, a follow-up testing plan is determined based on risk evaluation. In some implementations, a follow-up testing plan may be determined using the statistical model to determine when an additional test (or tests) should be conducted to reduce the risk value determined using the statistical model such that the risk value will satisfy the threshold. In some implementations, the follow-up testing plan is determined by modeling hypothetical negative test results occurring at different dates to determine hypothetical risk values until the hypothetical risk value is low enough to satisfy the threshold. In some implementations, a standard follow-up test plan may be determined in advance based on the statistical model rather than on a case-by-case basis (e.g., after a flight an individual must test negative on day 0 (the day of the flight) and day 6 to reduce the risk value below the threshold).

At operation 314, follow-up test results are received based on testing conducted according to the follow-up testing plan. For example, the individual may return to a testing station for another test. The results of this follow-up test may then be transmitted to the infection tracking system 102. The operation 314 may be performed similarly to the operation 306.

At operation 316, notice that an activity is permitted is transmitted. For example, the notice may be transmitted to the individual. The notice may indicate that a quarantine is complete. The notice may be transmitted as a text (SMS) message to a phone associated with the individual. The notice may also be transmitted to an administrator who is managing a quarantine or managing access to an event. The notice may be transmitted through an application that is running on a computing system.

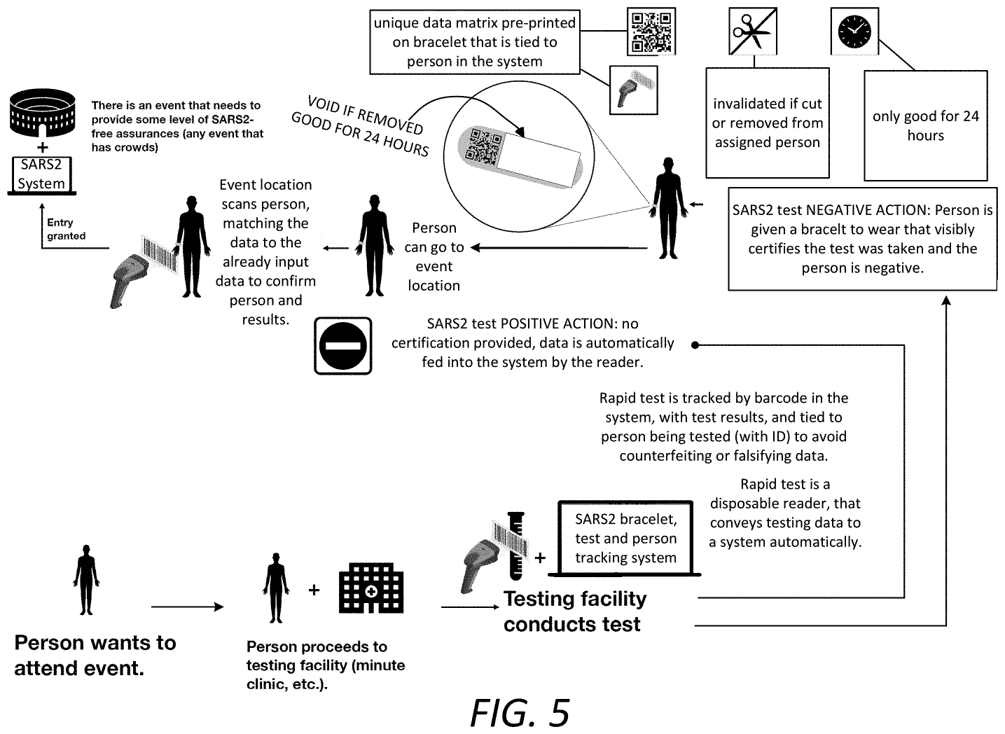

FIG. 5 illustrates an example method of tracking infection so that an individual may participate in a group event. In this method, the individual visits a testing center to receive a test. The results of the test are transmitted, by a computing device such as a smartphone, to another computing system over a network. In some embodiments, the identity of the individual is not associated with the test. Instead, the test results are associated with a unique identifier of the test (e.g., which may be included with a test kit). The computing device used to transmit the test results may include a specialized reader for reading the results of the test strip. The reader may be reusable or disposable (e.g., configured for one-time use).

In some embodiments, a biometric identifier of the individual may be captured and transmitted along with the test results. For example, a retinal image or scan may be captured and associated with the test. The retinal image or scan may be captured with a photograph, which may be taken using a smartphone used to transmit the test results. In some implementations, the photograph may be taken while the test is being administered and may include the individual's retina and an identifier of the test (e.g., a QR code or code on a swab that is used to gather sample from the patient). Other biometric values may also be captured for the individual too. In some embodiments, a temperature reading from the individual is taken and transmitted along with the test results. If the temperature indicates that the individual has a temperature, the test results may be considered inconclusive and may result in denial of admission regardless of the results of the lateral flow assay. Similarly, location information about where the test was performed (e.g., as determined by a GPS chip associated with the computing device transmitting the test results).

Temperature readings may include a numeric temperature value taken via a temporal scan, orally, or otherwise. Temperature readings may also include multiple temperature readings generated from one or more infrared images (e.g., a static image or a video). In some implementations, the temperature readings include thermal signatures obtained using image processing or artificial intelligence techniques on the one or more infrared images. The thermal signatures may be based on the temperature values and variations at multiple locations on an individual's face, head, or body. The thermal signatures may be usable to differentiate between normal temperature variations in healthy (non-infected) individuals and temperature variation caused by SARS-CoV-2 infection.

The individual is then given a wearable identifier such as a bracelet or anklet, which may be placed on the individual by the test administrator. In some embodiments, the wearable identifier may include components (e.g., a lock) that make it difficult for the individual to remove without additional equipment. In some embodiments, the wearable identifier includes tamper resistant technology such that it will be difficult or impossible for an individual to remove the wearable identifier without damaging or altering the wearable identifier. Examples of tamper resistant technology include wristbands made from Tyvek® material or another plastic material with an adhesive region that includes die cuts. Once adhered to an individual, removing the wristband would destroy the adhesive region, making it readily apparent if a wristband has been removed or transferred to another individual.

The wearable identifier includes a unique identifier that can be used to retrieve the associated test results or a corresponding status value. The status value may correspond to the likelihood that the individual associated with the unique identifier is at a specific time a contagious carrier of SARS-CoV-2. The status value may, for example, be based on a risk value determined by the risk assessment engine 124. In some embodiments, a status value of ADMIT may be provided for a specific duration of time following a negative SARS-CoV-2 test to indicate the unique identifier is associated with an individual who is unlikely be a contagious carrier of SARS-CoV-2. In contrast, a status value of DO NOT ADMIT may be provided to indicate that the unique identifier is not currently associated with test results indicating the individual is non-contagious.

For example, prior to admitting the individual to a location or event, an administrator may scan a computer readable code (e.g., a QR code, a barcode, etc.) on the wearable identifier to retrieve the status value to confirm that the individual tested negative and the test results are valid. The test results or status value may be considered valid for a predetermined time period (e.g., 24 hours).

The individual can then attend a group event (e.g., an event that would not be possible to attend with social distancing policies) for that time period following a negative test result. Similar processes may be used before or after air travel, use of mass transit, granting access to certain facilities, etc. In some implementations, the wearable identifier may include a test strip.

In some implementations, the duration of validity for the status value may be based on combining multiple different types of test results or temperature readings, which is sometimes referred to as test stacking. For example, a status value for an individual who has a negative test for SARS-CoV-2 but a positive serological test for SARS-CoV-2 antibodies (e.g., indicating a previous infection with SARS-CoV-2) may have a longer duration of validity (e.g., 2 weeks) than a status value for an individual who has simply had a negative test for SARS-CoV-2 (e.g., 24 hours). This difference in duration may reflect the reduced likelihood that an individual who has SARS-CoV-2 antibodies and has presumably recovered from a SARS-CoV-2 infection will be re-infected. The length of the additional duration may be based on if and for how long SARS-CoV-2 antibodies provide complete or partial immunity to re-infection.

In some embodiments, an individual's test results may be associated with information about the testing materials or equipment used, and the duration of validity may vary based on those materials or equipment. For example, tests performed with equipment that has a higher specificity or sensitivity may result in a status value that has a longer duration of validity. Furthermore, if information about the materials or equipment comes to light after a test has been completed, the duration of validity may be adjusted accordingly. For example, if after a test is completed, it is discovered that a specific batch of testing materials for a serological test is defective, the status values for any associated unique identifiers may be changed accordingly.

An example method for providing admission includes retrieving a test identifier from an individual. The test identifier may be received by scanning an identifier code on a wearable identifier worn by the individual. The method also includes retrieving an infection status indicator based on the test identifier. Retrieving the infection status indicator may include transmitting a request, by a computing device, over a network, to another computing device and receiving a response that includes the infection status indicator. The request may include the test identifier. The response may indicate that the test identifier is associated with a valid negative test result. The response may indicate that the test identifier is associated with a test result that is no longer valid for admission (e.g., the test results indicate a current infection, or the test result has expired due to the passage of time and the test results can no longer be relied upon to indicate that the individual is infection free). In some embodiments, the response may also indicate that the test identifier is associated with a positive test result. The method may also include admitting or denying access to the individual based on the received test result.

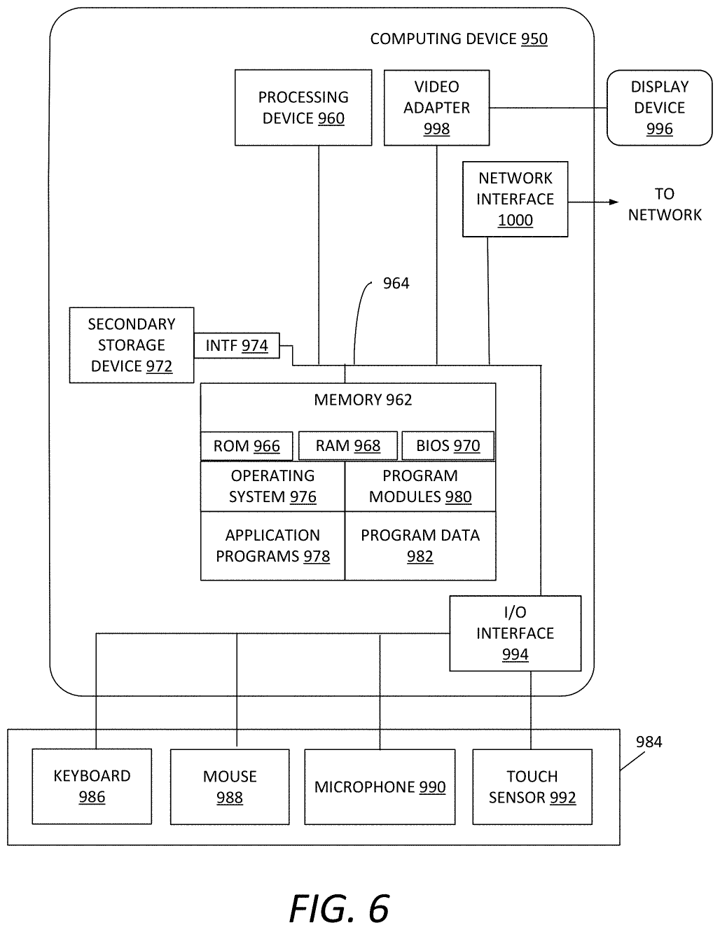

FIG. 6 illustrates an example architecture of a computing device 950 which can be used to implement aspects of the present disclosure, including any of the plurality of computing devices described herein, such as a computing device for uploading test results, retrieving test results, and deciphering test strip results in some embodiments, or any other computing devices that may be utilized in the various possible embodiments.

The computing device illustrated in FIG. 6 can be used to execute the operating system, application programs, and software modules described herein.

The computing device 950 includes, in some embodiments, at least one processing device 960, such as a central processing unit (CPU). A variety of processing devices are available from a variety of manufacturers, for example, Intel or Advanced Micro Devices. In this example, the computing device 950 also includes a system memory 962, and a system bus 964 that couples various system components including the system memory 962 to the processing device 960. The system bus 964 is one of any number of types of bus structures including a memory bus, or memory controller; a peripheral bus; and a local bus using any of a variety of bus architectures.

Examples of computing devices suitable for the computing device 950 include a desktop computer, a laptop computer, a tablet computer, a mobile computing device (such as a smartphone, an iPod® or iPad® mobile digital device, or other mobile devices), or other devices configured to process digital instructions.

The system memory 962 includes read only memory 966 and random-access memory 968. A basic input/output system 970 containing the basic routines that act to transfer information within computing device 950, such as during start up, is typically stored in the read only memory 966.

The computing device 950 also includes a secondary storage device 972 in some embodiments, such as a hard disk drive, for storing digital data. The secondary storage device 972 is connected to the system bus 964 by a secondary storage interface 974. The secondary storage devices 972 and their associated computer readable media provide nonvolatile storage of computer readable instructions (including application programs and program modules), data structures, and other data for the computing device 950.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory computer-readable media. Additionally, such computer readable storage media can include local storage or cloud-based storage.

A number of program modules can be stored in secondary storage device 972 or system memory 962, including an operating system 976, one or more application programs 978, other program modules 980 (such as the software engines described herein), and program data 982. The computing device 950 can utilize any suitable operating system, such as Microsoft Windows™, Google Chrome™ OS or Android, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 950 through one or more input devices 984. Examples of input devices 984 include a keyboard 986, mouse 988, microphone 990, and touch sensor 992 (such as a touchpad or touch sensitive display). Other embodiments include other input devices 984. The input devices are often connected to the processing device 960 through an input/output interface 994 that is coupled to the system bus 964. These input devices 984 can be connected by any number of input/output interfaces, such as a parallel port, serial port, game port, or a universal serial bus. Wireless communication between input devices and the interface 994 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 996, such as a monitor, liquid crystal display device, projector, or touch sensitive display device, is also connected to the system bus 964 via an interface, such as a video adapter 998. In addition to the display device 996, the computing device 950 can include various other peripheral devices (not shown), such as speakers or a printer.

When used in a local area networking environment or a wide area networking environment (such as the Internet), the computing device 950 is typically connected to the network through a network interface 1000, such as an Ethernet interface or WiFi interface. Other possible embodiments use other communication devices. For example, some embodiments of the computing device 950 include a modem for communicating across the network.

The computing device 950 typically includes at least some form of computer readable media. Computer readable media includes any available media that can be accessed by the computing device 950. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 950.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 6 is also an example of programmable electronics, which may include one or more such computing devices, and when multiple computing devices are included, such computing devices can be coupled together with a suitable data communication network so as to collectively perform the various functions, methods, or operations disclosed herein.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. An infection tracking system including:
an evaluation session management engine that is configured to generate an evaluation session data structure and associate the evaluation session data structure with an individual;
a test result processing engine that is configured to receive test results and associate the test results with the evaluation session data structure; and
a risk assessment engine that is configured to determine a risk level for an individual associated with an evaluation session data structure.

2. The infection tracking system of claim 1, wherein the evaluation session management engine is configured to associate the evaluation session data structure with the individual by storing identifying information about the individual.

3. The infection tracking system of claim 1, wherein the evaluation session management engine does not store any identifying information about the individual.

4. The infection tracking system of claim 3, wherein the evaluation session management engine is configured to associate the evaluation session data structure with the individual by storing a key or code that is associated with the individual.

5. The infection tracking system of claim 1, wherein the test result processing engine is configured to receive test results from a portable device that generates test results.

6. The infection tracking system of claim 1, wherein test result processing engine is configured to receive test results that include a binary value indicating the presence of infection.

7. The infection tracking system of claim 1, wherein the test result processing engine is configured to receive test results that include at least one of:
information about a type of test administered;
information identifying a manufacturer of test supplies or test equipment used to generate the test result;
a batch or serial number for the test supplies and test equipment used to generate the test results;
a location at which a test associated with the test results was administered;
a date when the test associated with the test results was administered; and
identification information associated with a test administrator.

8. The infection tracking system of claim 1, wherein the risk assessment engine is configured to determine the risk level for the individual based on a numeric risk value that corresponds to a likelihood that an individual who has been infected receives one or more negative test results.

9. The infection tracking system of claim 1, wherein the risk assessment engine is configured to determine the risk level for the individual based on retrieving activities associated with the individual associated with evaluations session.

10. The infection tracking system of claim 1, wherein the risk assessment engine is further configured to determine a quarantine duration for the individual.

11. The infection tracking system of claim 1, wherein the risk assessment engine is further configured to determine whether an individual may be admitted to participate in an activity based on comparing a determined risk level for the individual to a threshold for the activity.

12. The infection tracking system of claim 1, wherein the risk assessment engine is configured to determine the risk level based on at least one of:
a pre-contagious incubation period duration for a specific type of infection;
a duration of infectivity for the specific type of infection;
an infection prevalence for the specific type of infection; and
an effective reproduction rate for the specific type of infection.

13. The infection tracking system of claim 1, further including a notification engine that is configured to transmit notifications based on the determined risk level.

14. The infection tracking system of claim 13, wherein the notification engine that is configured to transmit notifications includes the notification engine being configured to transmit text messages to a phone number associated with the individual.

15. A method of evaluating risk of infection transmission, the method comprising:
receiving initial test results;
associating the initial test results with a session data structure;
evaluating a risk level based on the initial test results; and
comparing the risk level to a threshold.

16. The method of claim 15, further comprising:
receiving evaluation session initialization data, the evaluation session initialization data including information to associate the evaluation session with an individual.

17. The method of claim 16, further comprising:
determining a quarantine duration based on the comparing the risk level to the threshold;
receiving follow-on test results;
updating the risk level based on the initial test results and the follow-on test results;
comparing the updated risk level to the threshold; and
reducing the quarantine duration based on the comparing the updated risk level to the threshold.

18. The method of claim 16, further comprising:
responsive to the comparing the updated risk level to the threshold indicating that the threshold has been satisfied, transmitting a notice that an activity is permitted.

19. A non-transitory computer-readable storage medium comprising instructions stored thereon that, when executed by at least one processor, cause a computing system to perform a method comprising:
generating a session data structure;
associating the session data structure with an individual;
receiving initial test results;
associating the initial test results with the session data structure;
determining a risk level based on the initial test results and the session data structure;
comparing the risk level to a threshold;
responsive to determining the threshold has not been satisfied, determining a follow-on testing plan; and
transmitting a notification based on the follow-on test plan.

20. The non-transitory computer-readable storage medium of claim 19, further comprising:
receiving follow-on test results;
associating the follow-on test results with the session data structure;
determining an updated risk level based on the initial test results, the follow-on test results, and the session data structure;
comparing the updated risk level to the threshold; and
responsive to determining the threshold has been satisfied, transmitting a notification that an activity is permitted.
